(19) United States
(12) Patent Application Publication
(10) Pub. No.: US 2001/0020186 A1
(43) Pub. Date: Sep. 6, 2001

(54) Keyed intervertebral dowel

(76) Inventors: Todd M. Boyce, Aberdeen, NJ (US); Erik O. Martz, Jackson, NJ (US); Nelson L. Scarborough, Ocean, NJ (US)

Correspondence Address: Russell R. Kassner, Dilworth & Barrese, 333 Earle Ovington Blvd., Uniondale, NY, 11553, US

(21) Appl. No.: 09/745,643

(22) Filed: Dec. 22, 2000

Related U.S. Application Data
(63) Continuation of application No. 09/327,982, filed on Jun. 8, 1999, now abandoned.

(51) Int. Cl. A61F 2/44

(52) U.S. Cl. 623/17.16; 623/23.63; 606/61; 623/17.11

(57) ABSTRACT

An intervertebral implant having a tabbed configuration is provided. The intervertebral implant includes a substantially cylindrical body portion and a pair of radially extending tabs. The radially extending tabs may be provided as a single or double pair and may assume various shapes and configurations for engaging the interior of a bore formed between adjacent vertebrae. A throughbore or plurality of throughbores extend from the top surface of the implant to the bottom surface of the implant. The implant may be formed from a cortical ring cut from the diaphysis of a long bone by milling. Alternatively, the implant may be formed of any biocompatible material having the requisite strength requirements via any known process, i.e., molding. There is also disclosed a method of insertion of the implant including forming a stepped bore between adjacent vertebrae, inserting the implant between adjacent vertebrae with tabs in alignment with the spaced defined by the adjacent vertebrae and rotating the implant such that the tabs are rotated within an enlarged or stepped portion of the bore to secure it therein.

This application is a Continuation of U.S. patent application Ser. No. 09/327,982, which was filed Jun. 8, 1999, and is hereby incorporated herein by reference.

### BACKGROUND OF THE INVENTION

1. Technical Field

The present disclosure relates to an intervertebral implant for spinal fusion and more particularly, to an intervertebral dowel having at least two radially extending tabs for securing the dowel within a receiving bed formed in the intervertebral space.

2. Background of Related Art

The spine is a flexible column formed of a series of bone called vertebrae. The vertebrae are hollow and piled one upon the other, forming a strong hollow column for support of the cranium and trunk. The hollow core of the spine houses and protects the nerves of the spinal cord. The different vertebrae are connected together by means of articular processes and intervertebral, fibro-cartilages. In general, a vertebral body is made of a cortical shell enclosing a cancellous (spongy) bone core. The portion of the cortical bone shell facing the surface of the disk is the endplate.

The intervertebral fibro-cartilages are also known as intervertebral disks and are made of a fibrous ring filled with pulpy material. The disks function as spinal shock absorbers and also cooperate with synovial joints to facilitate movement and maintain flexibility of the spine. When one or more disks degenerate through trauma, spondylolisthesis or other pathologies, nerves passing near the affected area may be compressed and are consequently irritated. The result may be chronic and/or debilitating back pain. Various methods and apparatus, both surgical and non-surgical, have been designed to relieve such back pain.

One method designed to relieve such back pain is interbody spinal fusion. Typically, interbody spinal fusion involves distracting adjoining vertebrae of the spine so that the nerve root canal sizes are increased and nerve irritation is eliminated or reduced. In order to maintain the adjoining vertebrae in a distracted state, at least one intervertebral implant is inserted into a receiving bed formed between the vertebrae. The implant is positioned to engage the adjoining vertebrae to maintain the vertebrae at a fixed degree of distraction.

Preferably, the implant should stabilize the intervertebral space and become fused to adjacent vertebrae in order to prevent the implant and adjacent vertebrae from moving. The implant must also provide spinal load support between the vertebrae. Further, during the time it takes for fusion, i.e. biological fixation of the vertebrae, to be completed, the implant should have enough structural integrity to maintain the space without substantial degradation or deformation of the implant. The implant should also have sufficient stability to remain in place prior to actual completion of bone ingrowth fusion. The implant should include structure which maintains the implant in position between the vertebrae while bone ingrowth is occurring. To facilitate rapid bone growth, and thus quick fusion, the implant may include or be provided with a bone growth supporting material. Obviously, the material from which the implant is constructed should be a biocompatible material and, preferably, interact biologically with the body's own naturally occurring tissues.

A variety of different types of intervertebral implants have been developed to perform this function including spinal fusion cages, threaded bone dowels and stepped bone dowels. An exemplary implant is disclosed in U.S. patent application filed on even date herewith, under Certificate of Express Mail Label No. EL260888076US, and entitled “Ramp-Shaped Intervertebral Implant”, the entire disclosure of which is incorporated by reference herein.

Common deficiencies in some of the prior art implants may include expulsion of the implant from between adjacent vertebrae, difficulty in inserting the implant into position, and/or lack of ability to allow incorporation of implant into the body. Also, in some prior art spinal fusion methods utilizing implants, the vertebrae may need to be distracted to a large extent in order to position the implant between the vertebrae.

Accordingly, a need exists for an improved intervertebral implant which is configured to prevent the likelihood of expulsion or retropulsion during normal patient activity, provide ease of insertion and include structure to facilitate incorporation of the implant into the body. Furthermore, need exists for an improved intervertebral implant which can be inserted between vertebrae without excessive distraction of the vertebrae and a method of installing such an implant.

### SUMMARY

In accordance with the present disclosure, an intervertebral implant having tabbed securing structure is provided. The intervertebral implant includes a substantially cylindrical body portion and at least one pair of radially extending tabs that are configured to engage vertebral bodies.

By engaging the vertebrae, the tabs reduce the likelihood that expulsion or retropulsion might occur. This is particularly significant in that where an implant is pushed out of place, damage to vital structures including neural (the spinal cord and existing nerve roots) and vascular (the aorta and inferior vena cava) can occur resulting in possible injury or death. Additionally, the tabs assist in preventing migration of the implant due to rotation of the adjacent vertebrae.

The tabs may take the form of various shape and constructions, such as, for example, smooth rounded, wedge shaped, cam shaped, toothed, or threaded, etc. In alternate embodiments, two diametrically opposed pairs of tabs are provided on the cylindrical body portion. In various embodiments, a throughbore or a plurality of throughbores extend from a top surface of the implant to the bottom surface of the implant providing a space for boney bridging to occur between the vertebrae which are intended to be fused. The throughbore(s) is dimensioned to receive growth factors or other grafting materials to stimulate bone healing. The pairs of tabs may be provided adjacent the opening of the throughbore or may be offset 90° from the openings of the throughbore. In one embodiment of an intervertebral implant, the cylindrical body portion is tapered.

In an alternate embodiment, the implant has an abbreviated body portion and does not include a throughbore.

In another embodiment, the tabs are formed by inserting a cortical plug through the throughbore. Preferably, the cylindrical body portion includes a slot formed in one end thereof for receipt of an insertion tool and a bore extending between the slot and into the throughbore for facilitating insertion and facilitating injection into the throughbore of any desirable material, such as, for example, bone growth stimulants, autograft, allograft, demineralized bone matrix, or other bone grafting materials.

Further, alternate embodiments may include body portions having shapes other than cylindrical, such as, those having rectangular, oval, multi-sided, etc., cross-sections.

In a preferred embodiment, the implant is formed from a cortical ring allograft cut from the diaphysis of a long bone. By utilizing bone or bone-derived materials as the implant material, the implant has the added advantage of facilitating incorporation of the implant into the body. The implant can be formed by milling the top and bottom surfaces of a cortical ring to form the substantially cylindrical body portion and a pair of radially extending wings. The implant is further milled such that the radially extending wings are formed into tabs each of which is spaced a predetermined distance from the end of the cylindrical body portion. Additionally, each tab may be milled so as to form the desired camming, wedge, threaded, etc. shape. The implant is milled such that the intramedullary canal of the cortical ring defines a throughbore in the cylindrical body portion of the implant. Alternatively, the implant may be formed of any biocompatible material such as titanium and titanium alloys, stainless steel, carbon fiber, ceramics, etc. having the requisite strength requirements via any known process, i.e., molding, machining, etc. Further, it is preferable that the implants be surface demineralized prior to use by exposing them to acid or other demineralizing solutions.

Preferably, the bone should be surface demineralized prior to use. Where partially or surface demineralized bone is utilized, such bone can be obtained employing known demineralization techniques, e.g., those employing strong acids such as hydrochloric acid as described in Reddi et al., Proc. Nat. Acad. Sci. 69, pp. 1601-1605 (1972), the entire disclosure of which is incorporated herein by reference. The extent of demineralization is a function of the strength of the acid solution, the shape of the bone and the duration of the demineralization treatment as disclosed in Lewandrowski et al., J. Biomed. Materials Res., 31, pp. 365-372 (1996) the disclosure of which is incorporated by reference herein. The use of partially or surface demineralized bone is beneficial since such substances exhibit greater initial osteogenic and/or osteoinductive activity than fully mineralized bone.

There is also disclosed a method of inserting the tabbed implant between adjacent vertebrae. The method involves forming a stepped bore between adjacent vertebrae, providing an intervertebral implant having a cylindrical body portion and at least one pair of diametrically opposite radially extending tabs extending from the cylindrical body portion and inserting the implant between adjacent vertebrae such that the tabs are in alignment with the space defined between adjacent vertebrae. The method further includes positioning the implant such that the tabs are within the enlarged areas of the stepped bore and rotating the implant such that the tabs enter the enlarged or stepped area of the bore. This provides a greater ease of insertion over other styles of implants, such as, for example, threaded implants.

### BRIEF DESCRIPTION OF THE DRAWINGS

Various preferred embodiments are described herein with reference to the drawings wherein:

FIG. 1 is a perspective view of one embodiment of the presently disclosed intervertebral implant;

FIG. 2 is a side view of the intervertebral implant shown in FIG. 1;

FIG. 3 is a top view of the intervertebral implant shown in FIG. 1;

FIG. 4 is a front view of the intervertebral implant shown in FIG. 1;

FIG. 5 is a side view of a long bone;

FIG. 6 is a perspective view of a ring cut from the long bone shown in FIG. 5;

FIG. 7 is a side view of the ring shown in FIG. 6;

FIG. 8 is a perspective view of the ring after the top surface has been milled;

FIG. 9 is a perspective view of the ring after the bottom surface has been milled;

FIG. 10 is a perspective view of the ring after the side walls have been machined;

FIG. 11 is a perspective view of the ring after the radially extending wings have been machined to form tabs;

FIG. 12 is a an end view of the vertebral space with a stepped hole drilled therein;

FIG. 13 is a side view of the vertebral space shown in FIG. 12;

FIG. 14 is an end view of the vertebral space of FIG. 12 with one embodiment of the presently disclosed intervertebral implant inserted therein;

FIG. 15 is a perspective view similar to FIG. 14 with the intervertebral implant rotated 90°;

FIG. 16 is a side view of the intervertebral space similar to FIG. 13 with the intervertebral implant inserted and rotated 90°;

FIG. 17 is a perspective view of another embodiment of the presently disclosed intervertebral implant;

FIG. 18 is a side view of the intervertebral implant shown in FIG. 17;

FIG. 19 is a top view of the intervertebral implant shown in FIG. 17;

FIG. 20 is a front view of the intervertebral implant shown in FIG. 17;

FIG. 21 is a perspective view of another embodiment of the presently disclosed intervertebral implant;

FIG. 22 is a side view of the intervertebral implant shown in FIG. 21;

FIG. 23 is a top view of the intervertebral implant shown in FIG. 21;

FIG. 24 is a front view of the intervertebral implant shown in FIG. 21;

FIG. 25 is a perspective view of another embodiment of the presently disclosed intervertebral implant;

FIG. 26 is a side view of the intervertebral implant shown in FIG. 25;

FIG. 27 is a top view of the intervertebral implant shown in FIG. 25;

FIG. 28 is a front view of the intervertebral implant shown in FIG. 25;

FIG. 29 is a perspective view of another embodiment of the presently disclosed intervertebral implant;

FIG. 30 is a side view of intervertebral implant shown in FIG. 29;

FIG. 31 is a to view of the intervertebral implant shown in FIG. 29;

FIG. 32 is a front view of the intervertebral implant shown in FIG. 29;

FIG. 33 is a perspective view of another embodiment of the presently disclosed intervertebral implant;

FIG. 34 is a side view of the intervertebral implant shown in FIG. 33;

FIG. 35 is a top view of the intervertebral implant shown in FIG. 33;

FIG. 36 is a front view of the intervertebral implant shown in FIG. 33;

FIG. 37 is a perspective view of another embodiment of the presently disclosed intervertebral implant;

FIG. 38 is a side view of the intervertebral implant shown in FIG. 37;

FIG. 39 is top view of the intervertebral implant shown in FIG. 37;

FIG. 40 is a front view of the intervertebral implant shown in FIG. 37;

FIG. 41 is a perspective view of another embodiment of the presently disclosed intervertebral implant;

FIG. 42 is a side view of the intervertebral implant shown in FIG. 41;

FIG. 43 is a top view of the intervertebral implant shown in FIG. 41;

FIG. 44 is a front view of the intervertebral implant shown in FIG. 41;

FIG. 45 is a perspective view of another embodiment of the presently disclosed intervertebral implant;

FIG. 46 is a side view of the intervertebral implant shown in FIG. 45;

FIG. 47 is a top view of the intervertebral implant shown in FIG. 45;

FIG. 48 is a front view of the intervertebral implant shown in FIG. 45;

FIG. 49 is a perspective view of another embodiment of the presently disclosed intervertebral implant;

FIG. 50 is a side view of the intervertebral implant shown in FIG. 49;

FIG. 51 is a top view of the intervertebral implant shown in FIG. 49;

FIG. 52 is a front view of the intervertebral implant shown in FIG. 49;

FIG. 53 is a perspective view of another embodiment of the presently disclosed intervertebral implant;

FIG. 54 is a side view of the intervertebral implant shown in FIG. 53;

FIG. 55 is a top view of the intervertebral implant shown in FIG. 53;

FIG. 56 is a front view of the intervertebral implant shown in FIG. 53;

FIG. 57 is a perspective view of another embodiment of the presently disclosed intervertebral implant;

FIG. 58 is a side view of the intervertebral implant shown in FIG. 57;

FIG. 59 is a top view of the intervertebral implant shown in FIG. 57;

FIG. 60 is a front view of the intervertebral implant shown in FIG. 57;

FIG. 61 is a perspective view of another embodiment of the presently disclosed intervertebral implant body portion with a rectangular cross-section;

FIG. 62 is a perspective view of another embodiment of the presently disclosed intervertebral implant body portion with an oval cross-section; and

FIG. 63 is a perspective view of another embodiment of the presently disclosed intervertebral implant body portion with a multi-sided cross-section.

### DETAILED DESCRIPTION OF PREFERRED EMBODIMENTS

Preferred embodiments of the presently disclosed intervertebral implant will now be described in detail with reference to the drawings, in which like reference numerals designate identical or corresponding elements in each of the several views.

The spinal interbody fusion devices or intervertebral implants according to the present disclosure are intended to be placed between adjacent vertebrae in an attempt to correct a debilitating degeneration of the spinal structure. In humans, the device may be used predominantly in the lumbar region of the spine, but is adjustable for use in the thoracic and cervical regions as well. When in place, the device supports and maintains an appropriate distance between vertebrae and causes bone tissue to form and become integral with the device. Consequently, the intervertebral space becomes filled with autologous bone tissue and forms an integral rigid bone construction between adjacent vertebrae. While the disclosed implants and methods are discussed in terms of humans, it is contemplated that the disclosed implants and methods may find beneficial use in veterinary applications.

The disclosed intervertebral implants are formed with a tabbed configuration which allows the implants to be inserted between the vertebrae and twisted or rotated to secure the implant in position between the vertebrae. This has the resultant benefits of reduced likelihood of expulsion. Furthermore, the implants disclosed herein also allow insertion of the implant between the vertebral space without excessive distraction between the vertebrae.

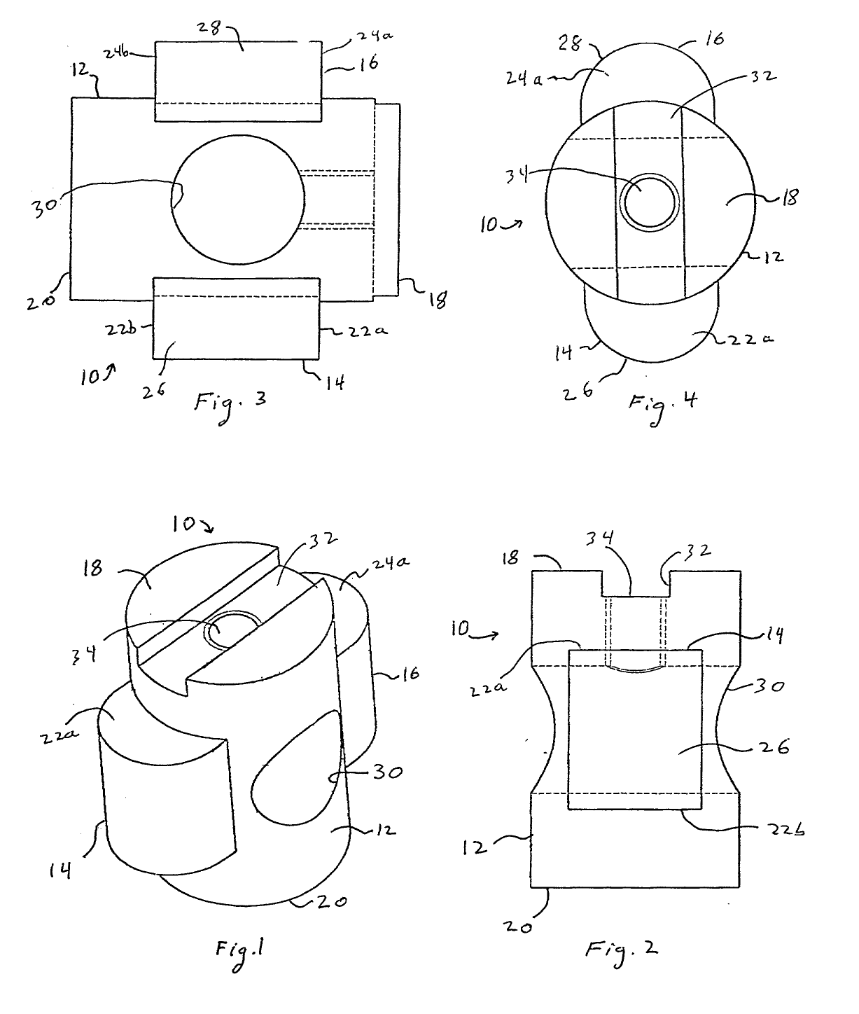

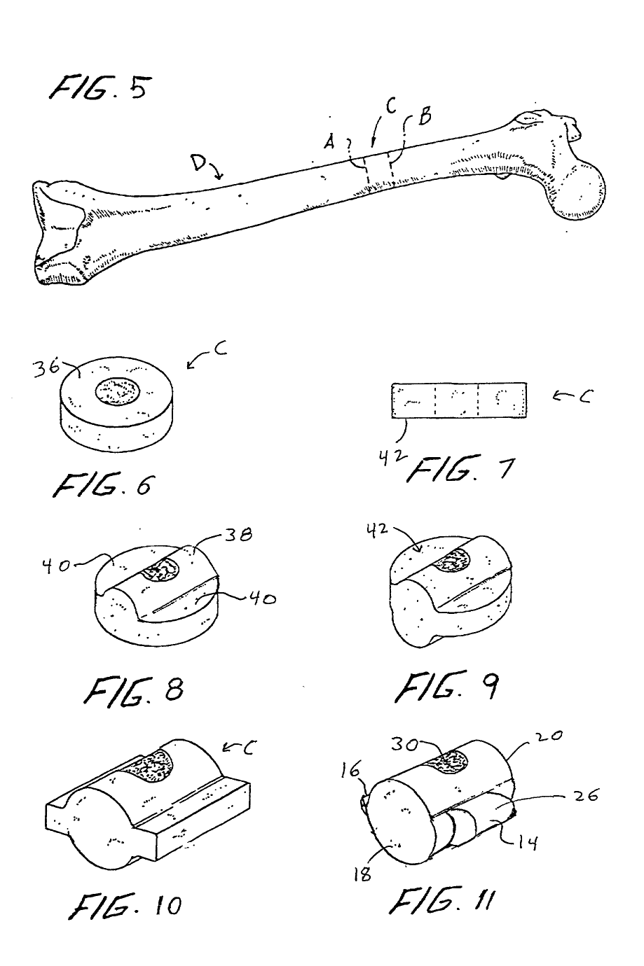

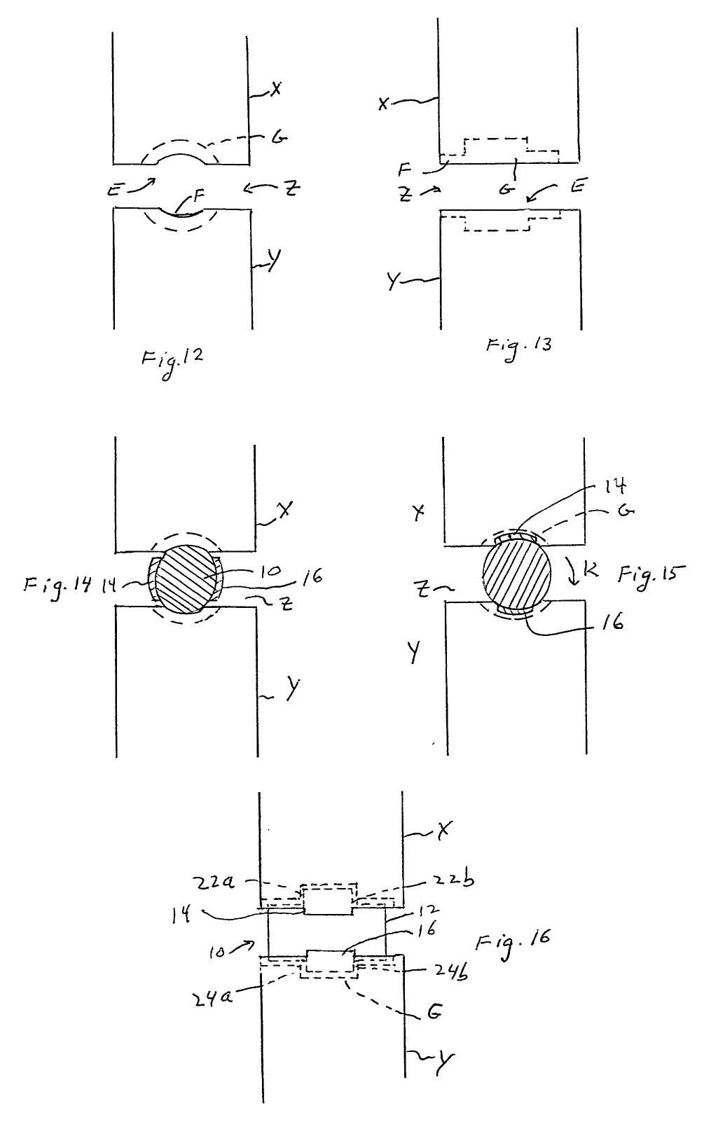

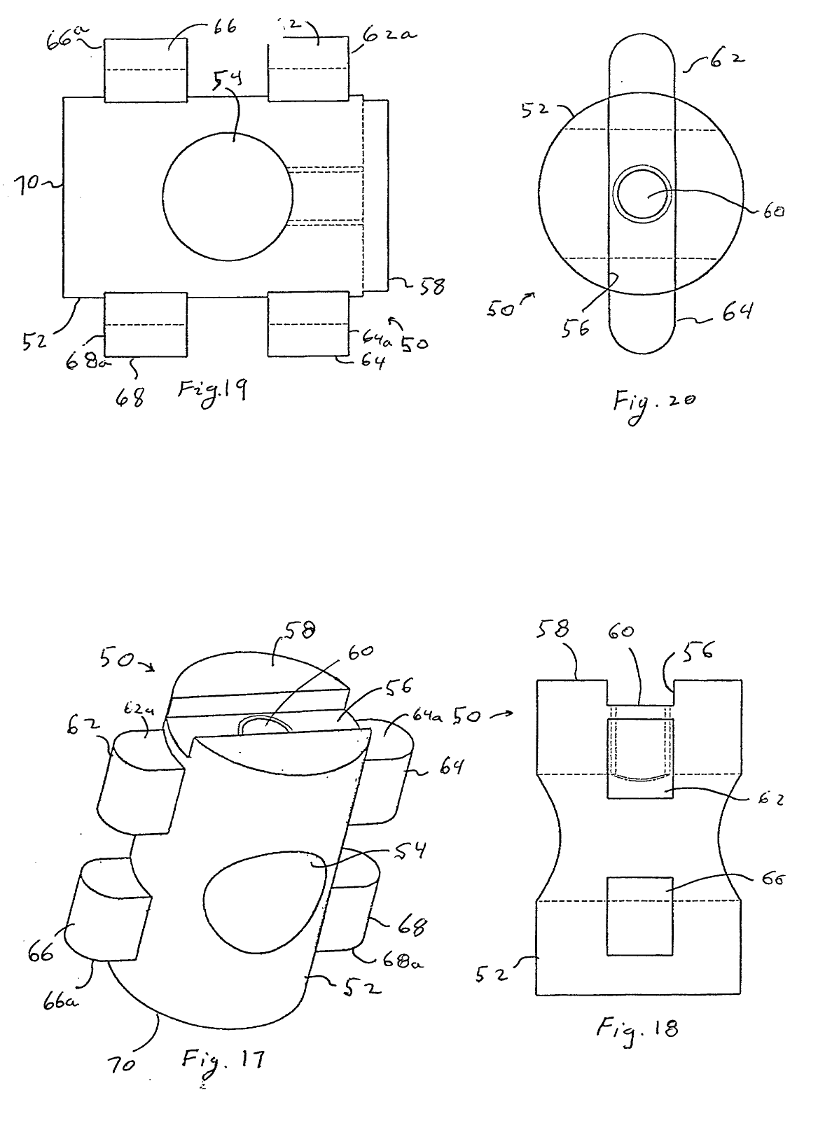

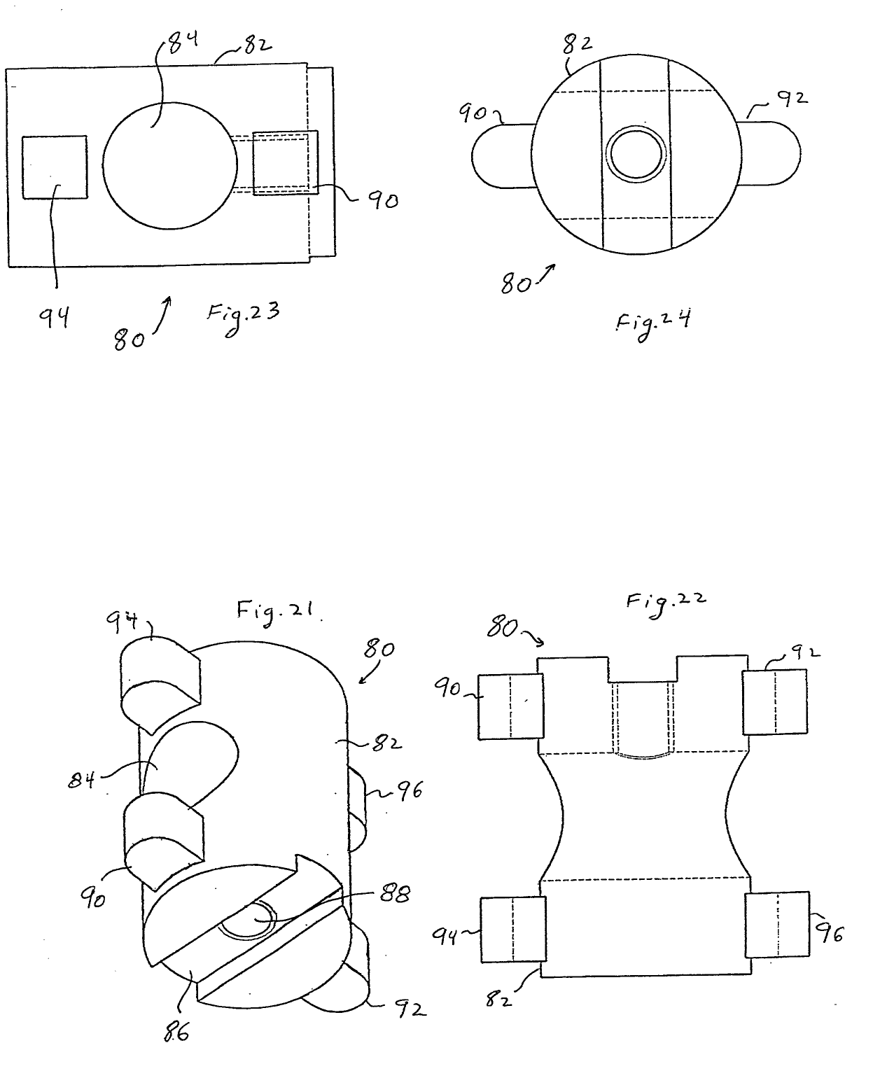

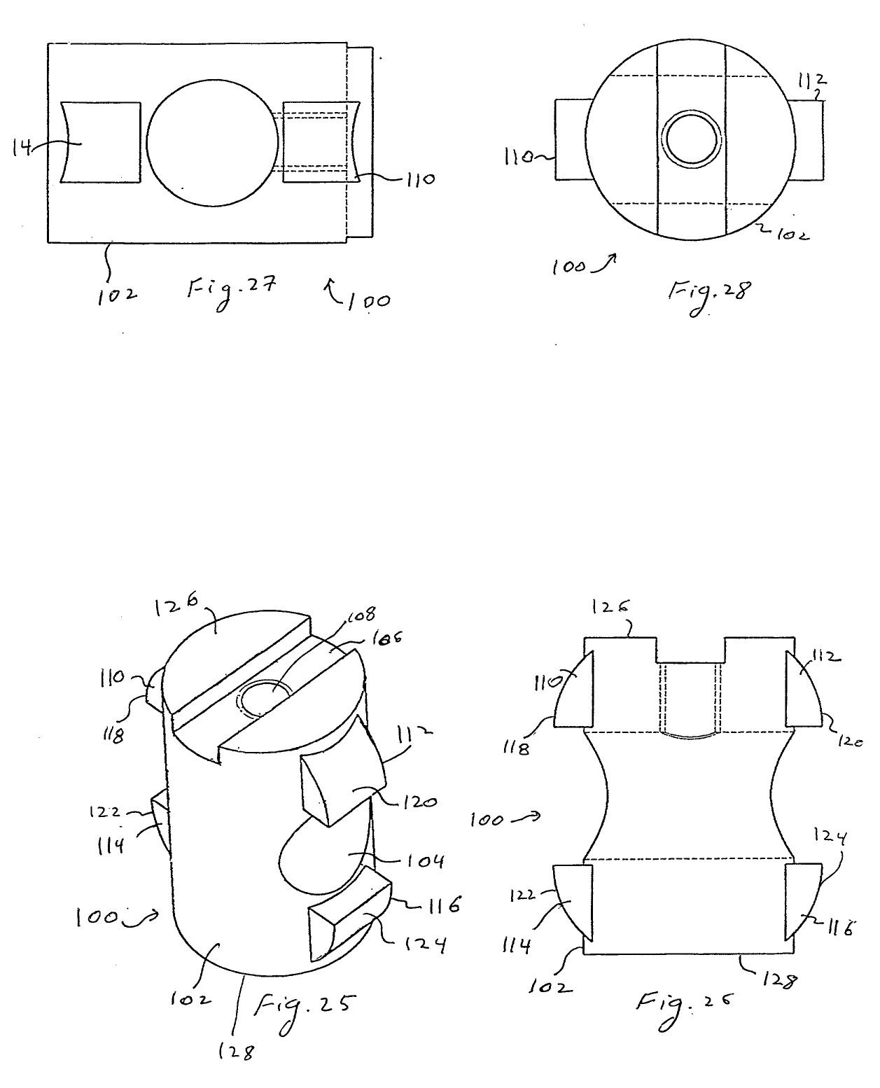

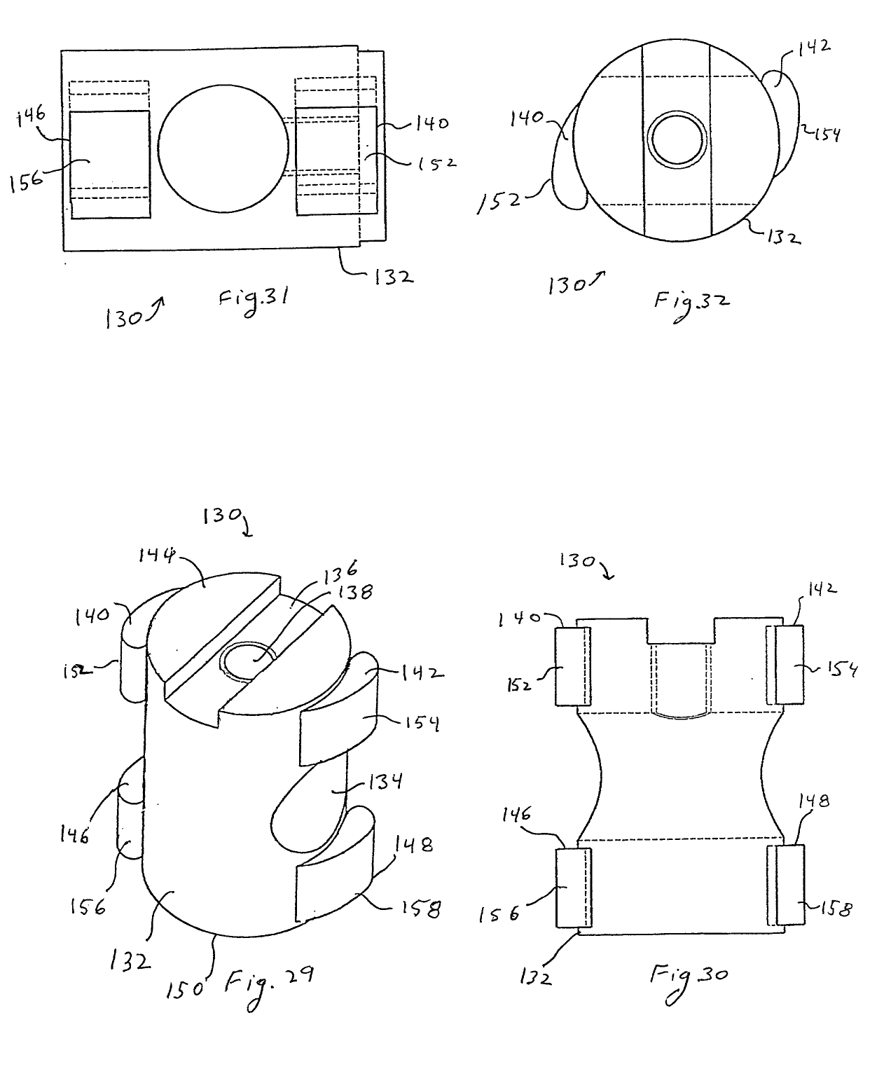

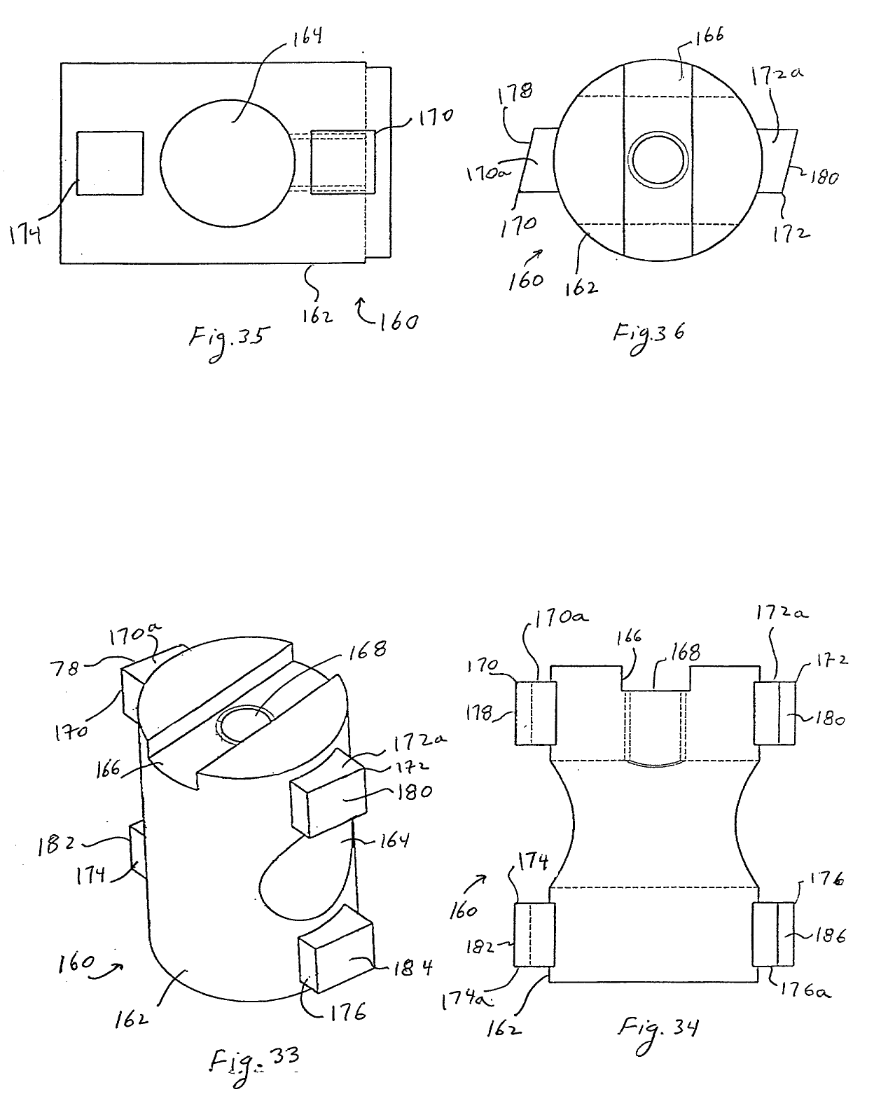

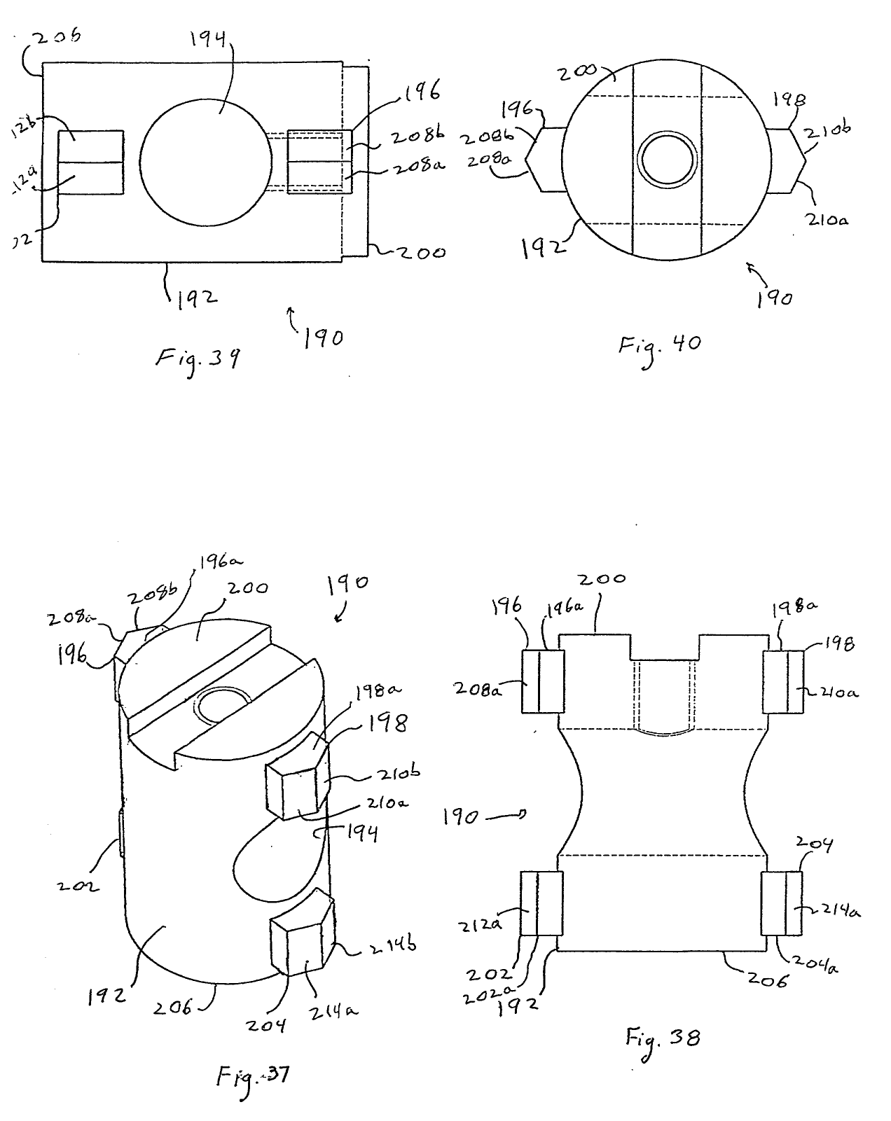

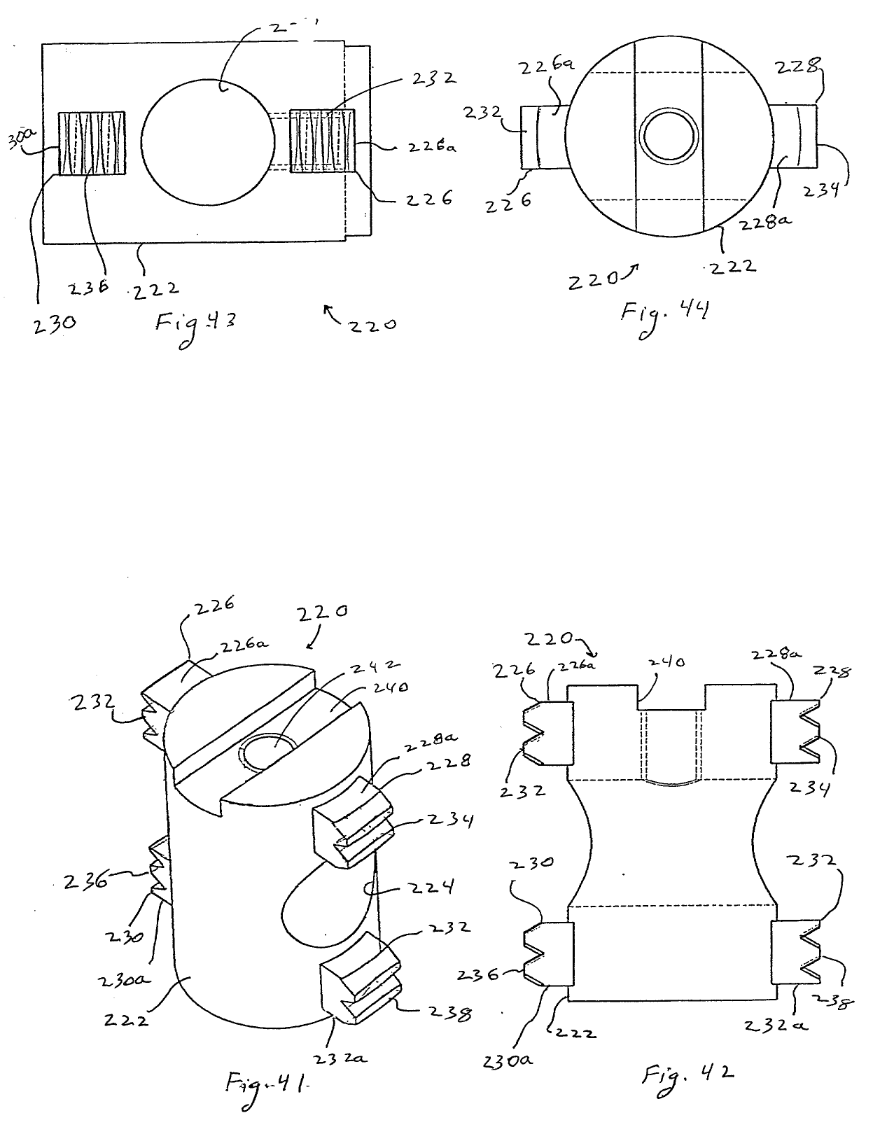

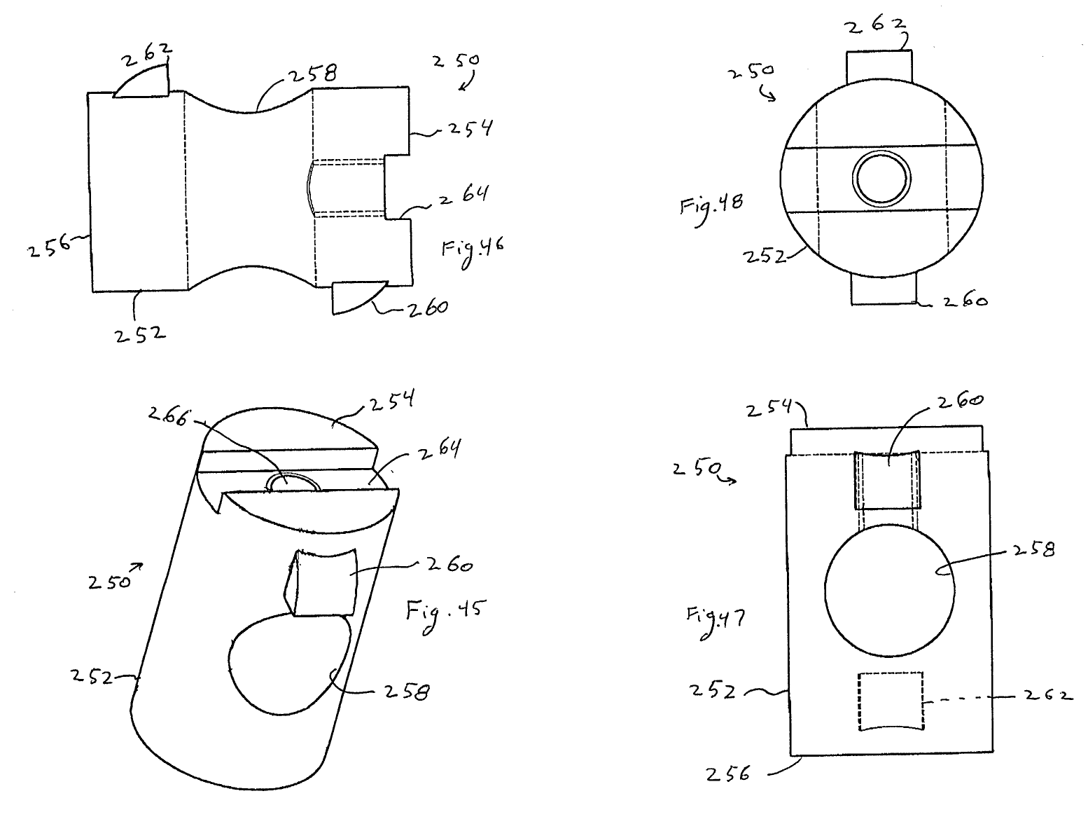

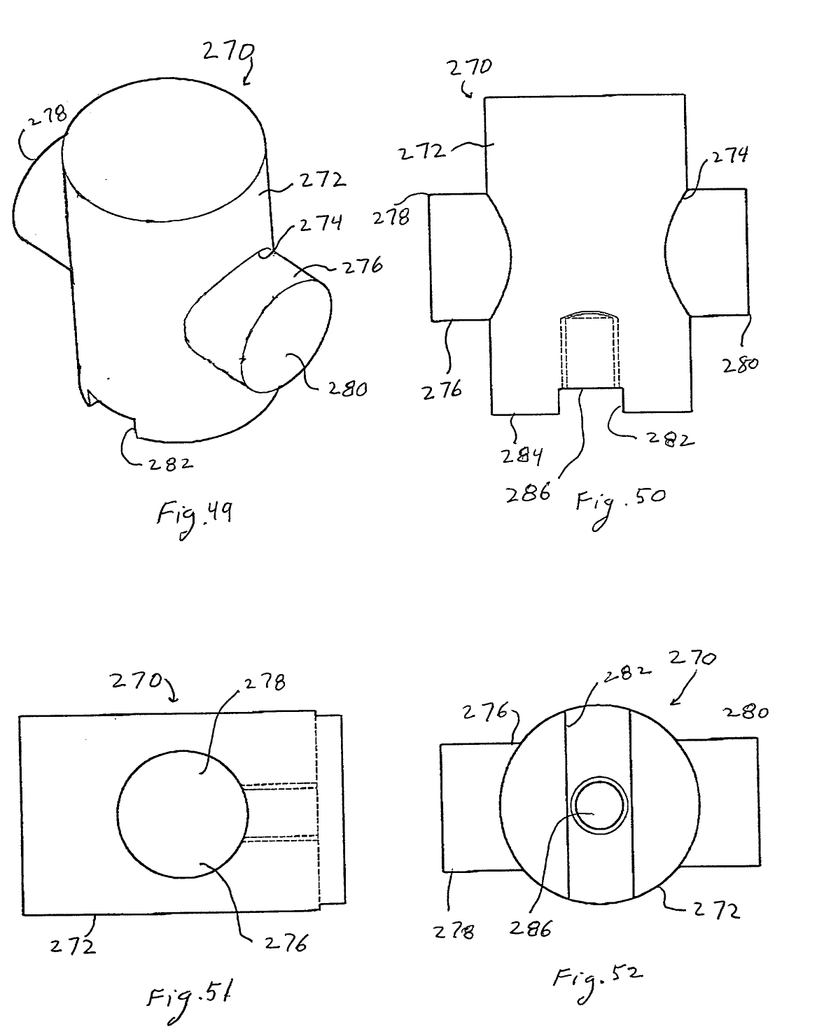

Referring now to FIGS. 1-4, there is illustrated one embodiment of the presently disclosed intervertebral implant shown generally as 10. Briefly, intervertebral implant 10 includes a substantially cylindrical body portion 12 having a pair of diametrically opposed and radially extending tabs 14 and 16. Cylindrical body portion 12.has a first end 18 and a second end 20. Tab 14 has first and second engaging or retaining surfaces 22a and 22b which are stepped or longitudinally spaced a predetermined distance from first end 18 and second end 20, respectively. Similarly, tab 16 has a pair of retaining surfaces 24a and 24b which are similarly stepped or longitudinally spaced from a first end 18 and second end 20 respectively. Retaining surfaces 22a, 22b and 24a, 24b are configured to engage a portion of adjacent vertebrae when installed therebetween.

As shown, tabs 14 and 16 extend only along a limited extent of the circumference of a cylindrical body portion 12. Preferably, tabs 14 and 16 are radially spaced 180° apart. Tab 14 includes a rounded side surface 26 and tab 16 includes a rounded side surface 28.

As shown, implant 10 includes a throughbore 30 which has a longitudinal axis substantially perpendicular to the longitudinal axis of implant 10. Further, implant 10 may be provided with perforations instead of, or in addition to, throughbore 30. Where implant 10 is formed of bone, the perforations assist in facilitating biological attachment and eventual incorporation of the implant into adjacent vertebrae.

Implant 10 further includes an installation slot 32 machined or milled in first end 18. A second bore 34 extends between slot 32 and throughbore 30. Second bore 34 is provided for mating of the implant with an insertion tool. Throughbore 30 is dimensioned to receive bone particles and/or biocompatible osteoinductive or osteoconductive material. These materials may include cancellous bone, cancellous bone particles, ceramics, polymers, composites, BMP, etc.

Intervertebral implant 10 can be constructed from a broad range of biocompatible materials such as, for example, surgical stainless steel, titanium, ceramic, hydroxyapatite, polymer, carbon fiber, tantalum, etc. Preferably, implant 10 is constructed from a human and/or animal cadaver bone. Intervertebral implant 10, appropriately sized, can be used in cervical, thoracic and lumbar spinal fusion procedures. For cervical spinal fusion procedures, in which implants are typically between 8 to 15 mm in length and 10 to 14 mm in diameter, bone is preferably obtained from the fibula, radius, ulna or humerus bones. For thoracic and lumbar spinal fusion procedures in which implants are typically 10 to 30 mm in length and/or diameter and about 10 to 14 mm in height, bone is preferably obtained from the humerus, femur or tibia. The sources of cortical bone for the bone-derived implant are preferably allogenic but also include xenogenic sources such as bovine and porcine bone.

Additionally, the bone may be subjected to penetration with osteogenic or demineralization agents during manufacture of the implant.

Alternatively, as discussed above, intervertebral implant 10 can be molded or machined from other biocompatible materials including composites made of bone as discussed in U.S. Pat. No. 5,899,939 to Boyce et al., the entire disclosure of which is incorporated by reference herein.

Referring now to FIGS. 5-11, in one preferred embodiment, intervertebral implant 10 is manufactured in accordance with the procedure disclosed in U.S. patent application filed on even date herewith under Certificate of Express Mail Label No. EL260888080US and entitled, “Intervertebral Implant”, the entire disclosure of which is incorporated by reference herein. In general, implant 10 is manufactured from a ring C formed by making transverse cuts through a long bone D along lines A and B as illustrated in FIG. 5. Next, the top 36 of ring C is machined using a milling device (not shown) having a dome or crown configuration to shape one side of ring C to have a semi-cylindrical portion 38 with two radially extending flats 40 (FIG. 8). Ring C is flipped over and the same milling procedure is formed on a bottom 42 of ring C as shown in FIG. 9. Next, the front and side surfaces are machined to flatten the side surface to reconfigure femoral ring C to have a generally rectangular configuration (FIG. 10). Finally, tabs 14 and 16 are formed by machine flats 40 so as to provide stepped surfaces from first and second ends 18 and 20 (FIG. 11). Additionally, further milling may be performed to provide rounded side surfaces 26 and 28 on tabs 14 and 16 respectively. It should be noted that throughbore 30 may be formed from a medullary canal through the long bone and further milled to provide a uniform throughbore 30 through ring C. While not shown, first end 18 may be further milled and/or drilled to provide installation slot 32 and bore 34 extending between installation slot 32 and an interior of throughbore 30. As discussed above, intervertebral implant 10 need not be formed from cadaveric bone but rather may be formed from any biocompatible material. As such, other known processes, such as molding techniques may be used to manufacture the implant.

Installation of implant 10 between a pair of adjacent vertebrae will now be described. Referring to FIGS. 12-16 and initially to FIGS. 12-13, there is illustrated a pair of adjacent vertebrae X and Y defining intervertebral space Z therebetween. The endplate is stronger bone than is the cancellous core. Thus, cuts in the vertebral bodies permit the tabs of the implant to extend past the endplate and into the softer bone beneath. A camming approach for some of the following disclosed embodiments of the implant tabs allows the cancellous bone to be compressed against the implant thereby providing additional frictional resistance against implant movement. A drill or other known devices and methods are utilized to form a stepped hole or bore E between the adjacent vertebrae preferably by milling or machining. Examples of such devices and procedures are disclosed in U.S. Pat. No. 5,445,639, the entire disclosure of which is incorporated by reference herein. Stepped hole E preferably has narrow diameter portion F adjacent the outer surface of the vertebrae and enlarged portion G interior to the vertebrae. In preparation for use, intervertebral implant 10 may be demineralized as discussed hereinabove and mounted on suitable installation devices.

Referring now to FIG. 14, once installed on an insertion device, intervertebral implant 10 is inserted between vertebrae X and Y such that tabs 14 and 16 are aligned with the intervertebral space Z. Intervertebral implant 10 is inserted into the drilled hole a sufficient distance such that tabs 14 and 16 align with the enlarged portion G of bore E. Implant 10 is subsequently rotated approximately 90° such that tabs 14 and 16 rotate into enlarged portion G. As noted above, retaining surfaces 22a and 22b on tab 14 and retaining surfaces 24a and 24b on tab 16 engage edges of enlarged portion G of bore E and prevent expulsion of the implant from between the adjacent vertebrae A and B. It should be noted that the entire procedure may be accomplished without any substantial or excessive distraction between adjacent vertebrae. While the present disclosure provides installation slot 37 and bore 32 for receipt of an installation device, it is within the contemplated scope of the present disclosure to provide implant 10 with other structure to allow insertion and rotation of the implant by various insertion tools.

Referring now to FIGS. 17-19, there is disclosed an alternative embodiment of an intervertebral implant. Intervertebral implant 50 is similar to implant 10 described above and generally includes cylindrical body portion 52 having a throughbore 54 formed therein. An installation slot 56 is provided in a first end 58 and a bore 60 extends from slot 56 to the interior of throughbore 54 similar to that described above with respect to implant 10.

Implant 50 includes a pair of radially extending first tabs 62 and 64 adjacent to, and longitudinally displaced from, first end 58 and a pair of second tabs 66 and 68 adjacent to, and longitudinally spaced from, a second end 70 of cylindrical body portion 52. Thus, first tabs 62 and 64 as well as second tabs 66 and 68 are stepped from first and second ends 58 and 70 respectively. First tabs 62 and 64 include engaging surfaces 62a and 64a for engaging an edge of stepped bore in a drilled vertebrae. Similarly, second tabs 66 and 68 also include engaging surfaces 66a and 68a for engaging an interior of a bore drilled in bone or vertebrae. Similar to that disclosed with regard to implant 10, first tabs 62 and 64 as well as second tabs 66 and 68 may have a generally rounded profile.

Intervertebral implant 50 is formed in the manner disclosed above with respect to implant 10 and is similarly installed in a stepped bore drilled in adjacent vertebrae. The stepped bore may have only a single enlarged area or may include two separate enlarged areas to accommodate the first and second tabs as the intervertebral implant is rotated into place.

Referring now to FIGS. 21-24, there is disclosed another alternate embodiment of an intervertebral implant similar to that of implant 50. Intervertebral implant 80 includes a generally cylindrical body portion 82 having a throughbore 84 formed therethrough. An installation slot 86 is provided along with a bore 88 extending between installation slot 86 and an interior of throughbore 84. Implant 80 includes a pair of radially extending first tabs 90 and 92 as well as a pair of radially extending second tabs 94, 96. In contrast to implant 50, first tabs 90, 92 and second tabs 94, 96 are formed on cylindrical body portion such that they are generally perpendicular to slot 86 and are adjacent to throughbore 84.

In the presently disclosed embodiments where the tabs are adjacent to the throughbore, a different method of forming the implant from bone is necessary. The bone will initially be cut parallel to the long axis of the long bone to permit the tabs to extend in a plane that transects the medullary canal. Subsequently, the presently disclosed methods of milling or machining the bone are performed to form the body portion and tabs. An installation shaft and bore between the installation slot and throughbore may be formed.

Referring now to FIGS. 25-28, there is disclosed another embodiment of an intervertebral implant which includes specific wedging structure to prevent the implant from moving longitudinally within a bore. Implant 100 generally includes a cylindrical body portion 102 having a throughbore 104 formed therein. Similar to previous embodiments, implant 100 is provided with an installation slot 106 and a bore 108 extending between installation slot 106 and throughbore 104. Implant 10 also includes a pair of radially extending first anterior tabs 110, 112 and a pair of radially extending second tabs 114, 116. As shown, first tabs 110 and 112 have curved wedge surfaces 118, 120. Similarly, second tabs 114 and 116 also include curved wedge surfaces 122 and 124. Wedge surfaces 118 and 120 of first tabs 110 and 112 curve away from a first end 126 of implant 10 and wedge surfaces 122, 124 of second tabs 114 and 116 curve away from a second end 128 of implant 100. The provision of wedge surfaces on the tabs provides a range of camming contact with the interior of a stepped bore drilled in adjacent vertebrae to thereby prevent expulsion of the implant.

Referring now to FIGS. 29-32, there is disclosed a further alternate embodiment of an intervertebral implant which includes progressive, radial camming structure which, upon rotation of the implant, cams the implant into position within a stepped bore. Specifically, intervertebral implant 130 includes a cylindrical body portion 132 having a throughbore 34 formed therethrough. An installation slot 136 may be provided along with a bore 138 extending between installation slot 136 and throughbore 134. Implant 130 additionally includes first tabs 140 and 142 formed adjacent first end 144 and second tabs 146 and 148 formed adjacent a second end 150. As illustrated, first tabs 140 and 142 as well as second tabs 146 and 148 have a generally, progressively curved shape such as a spline shape or one defined by a polynomial-defined curve. Thus, first tabs 140, 142 include progressive camming surfaces 152, 154. Second tabs 146 and 148 include progressive camming surfaces 156 and 158. Implant 130 may be formed in a manner similarly described above with respect to implant 10.

Upon installation of implant 130, between adjacent vertebrae, implant 130 is rotated and progressive camming surfaces 152, 154 and 156, 158 engage walls of the stepped bore in progressive fashion to firmly wedge implant 130 within the stepped bore and prevent any loosening or further rotation or reverse rotation of implant 130 within the stepped bore. The provision of progressive camming surfaces allows for the use of implant 130 in bores which may not have been drilled precisely or to a constant/consistent diameter. Further, as noted above, camming structure on the disclosed implants allows the tabs to compress the spongy bone to gain additional frictional force to secure the implant between the vertebrae.

Referring now to FIGS. 33-36, there is disclosed another alternate embodiment of an intervertebral implant including camming surfaces provided on tabs so as to allow the implant to be cammed within a stepped bore formed in adjacent vertebrae upon rotation of the implant. Specifically, implant 160 includes a cylindrical body portion having a throughbore 164 and installation slot 166 and a bore 168 extending between installation slot 166 and throughbore 164. A pair of radially extending first tabs 170, 172 and a pair of radially extending second tabs 174, 176 are formed on cylindrical body portion 162. First tabs 170 and 172 have relatively flat camming surfaces 178 and 180, respectively, formed thereon, while second tabs 174, 176 also include relatively flat camming surfaces 182, 184, respectively, formed thereon. As with implant 130, rotation of implant 160 within a stepped bore causes the camming surfaces 178, 180 and 182, 184 to engage sidewalls of the stepped bore and cam the implant therein to prevent further rotation. As with all prior embodiments, first tabs 170 and 172 also include camming surfaces 170a, 172a and second tabs 174, 176 include camming engaging surfaces 174a, 176a to engage edges of stepped bore and prevent expulsion of the implant after it has been rotated into position within the stepped bore.

Referring now to FIGS. 37-40, there is disclosed a further alternate embodiment of an intervertebral implant. Intervertebral implant 190 generally includes a cylindrical body portion 192 having a throughbore 194. Implant 190 includes first tabs 196 and 198 spaced a predetermined distance from first end 200 of cylindrical body portion 192. Implant 190 additionally includes second tabs 202 and 204 positioned adjacent and spaced a distance from second end 206 of cylindrical body portion 192. Implant 190 includes camming structure formed on the first and second tabs which permits rotation of the implant in either direction upon installation. Specifically, first tabs 196 includes opposed inclined camming surfaces 208a and 208b and first tab 198 also includes opposed inclined camming 210a and 210b. Similarly, second tab 202 includes opposed inclined camming surfaces 212a and 212b and second tab 204 includes opposed inclined camming surfaces 214a and 214b. The opposed inclined camming surfaces allow the implant to be rotated in either direction and still achieve a camming function within a stepped bore. As with prior embodiments, first tabs 196 and 198 include bore engaging surfaces 196a and 198a respectively. Similarly, second tabs 202 and 204 include bore engaging surfaces 202a and 204a respectively. Implant 190 may preferably be provided with an installation slot 216 and a bore 218 extending between slot 216 and throughbore 194.

Referring now to FIGS. 41-44, there is disclosed a further alternate embodiment of an intervertebral implant. Implant 220 generally includes cylindrical body portion 222 having a throughbore tube 224 defined therein. First tabs 226 and 228 and second tabs 230 and 232 extend radially from cylindrical body portion 222. The first and second tabs of implant 220 include threaded structure which allows the implant to engage precut threads in a stepped bore formed between adjacent vertebrae or to act as teeth to cut into bone and thereby secure implant 220 within a stepped bore between adjacent vertebrae. Alternatively, the tabs may be grooved but not necessarily threaded. Specifically, first tab 226 includes a threaded surface 234 and first tab 228 includes a threaded surface 236. Similarly, second tab 230 includes a threaded surface 238 and second tab 232 includes a threaded surface 240. It should be noted that the number of threads on any individual tab may differ from the number on an adjacent or diametrically opposed tab. Preferably, an installation slot 242 is provided having a bore 244 extending between slot 242 and into throughbore 224.

Referring now to FIGS. 45-48, there is disclosed an asymmetrical embodiment of an intervertebral implant. Implant 250 generally includes a cylindrical body portion 252 having a first end 254 and a second end 256. A throughbore 258 extends through implant. A first tab 260 is provided a predetermined spaced distance from first end 254 and a second tab 262 is provided a predetermined spaced distance from second end 256. As shown, first and second tabs 260, 262 are radially spaced approximately 180°. First and second tabs 260, 262 may be of any of the previously described shapes in the prior embodiments and include respective camming and/or abutment bone engaging surfaces. Additionally, implant 250 may be provided with an installation slot 269 and a bore 266 and be formed in accordance with the previously described methods and of same or similar materials.

Referring now to FIGS. 49-52, there is disclosed an intervertebral implant 270 designed to utilize a plug, which may be formed from cortical bone, to form the tabs. Implant 270 generally includes a cylindrical body portion 272 formed in accordance with the above described method such that the medullary canal provides a throughbore 274 in implant 270. A cortical plug 276 formed by turning on a lathe, milling, or other appropriate machining process. Plug 276 is positioned within throughbore 274 which may be suitably drilled or otherwise prepared to receive plug 276 such that first and second ends 278, 280 of cortical plug 276 extend radially outward from body portion 272. First and second ends 278, 280 thus form tabs which, when installed by the above described method, engage edges of a stepped bore formed in adjacent vertebrae. An installation slot 282 may be formed in an end 284 of body portion and a bore 286 extends between slot 282 and throughbore 274.

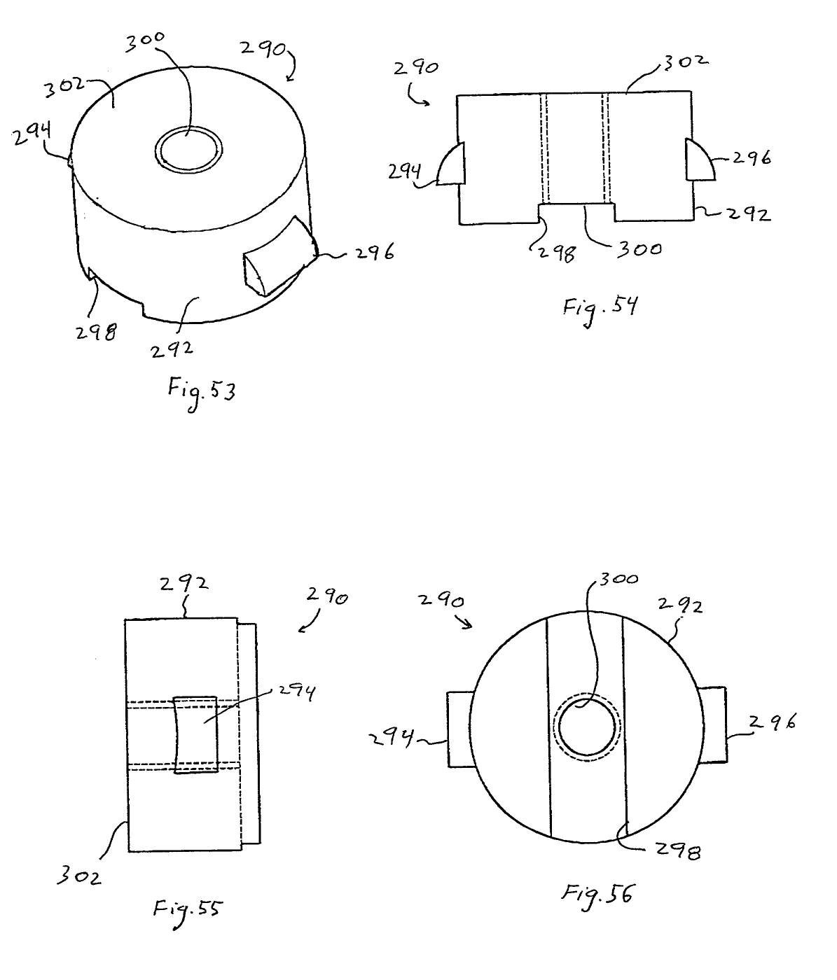

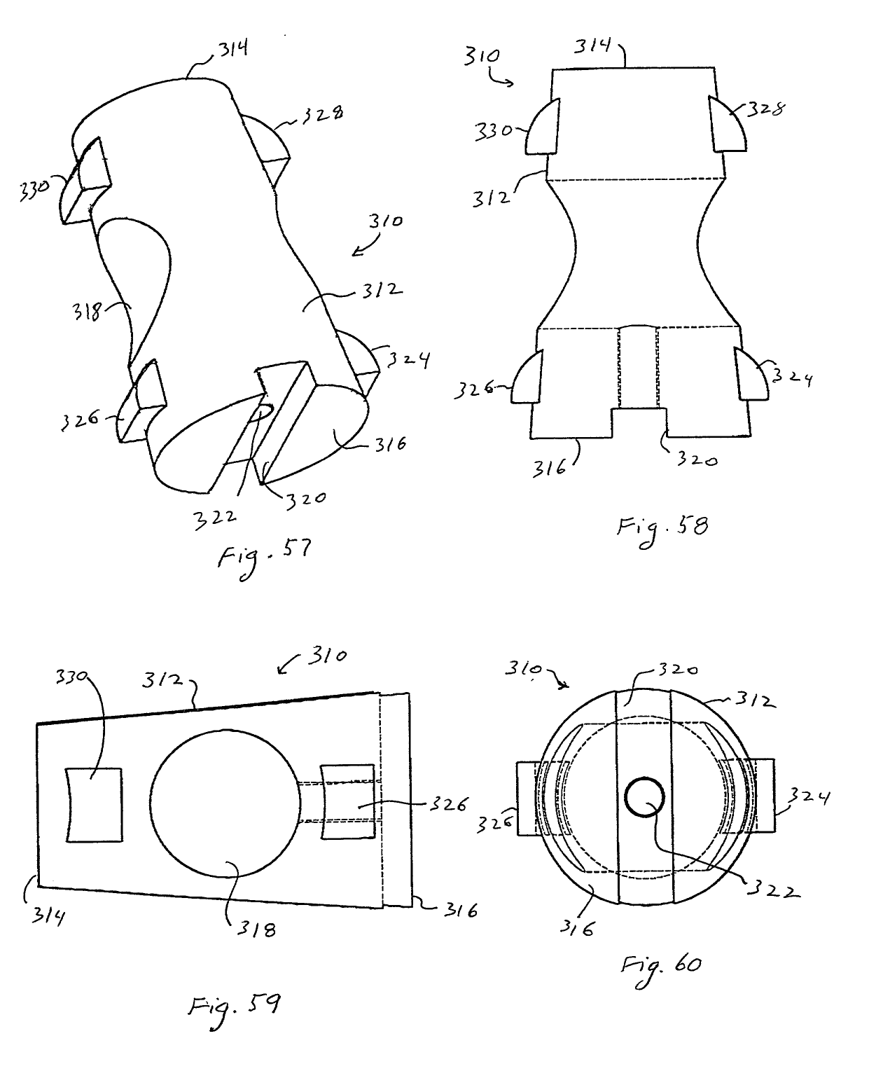

Referring now to FIGS. 53-56, there is disclosed an alternate embodiment of an intervertebral implant with a substantially shortened body portion. Implant 290 is designed to be provided in various diameters such that two or more implants 290 of differing diameters may be used together to introduce the appropriate lordosis into the spine. Implant 290 generally is similar to the above described implants except that the length of a cylindrical body portion 292 is substantially abbreviated or shortened. Implant 290 may include any of the previously described versions of tabs and preferably first and second tabs 294, 296. Implant 290 may also include an installation slot 298 and bore 300 extending between slot 298 and end face 302 of body portion 292. However, it is not contemplated that implant 290 have a throughbore and thus implant 290 may be formed from bone extending up to, but not including, the medullary canal of a long bone. Further, various body portion configurations, such as, for example, tapered, semi-conical, etc. are also envisioned.

Referring now to FIGS. 57-60, there is disclosed another embodiment of an intervertebral implant. Implant 310 generally includes a tapered cylindrical body portion 312 having a first end 314 and a second end 316. The diameter of first end 314 is smaller than the diameter of second end 316. Implant 310 may be formed by the disclosed method and include a throughbore 318, an installation slot 320 and a bore 322 extending from slot 320 to throughbore 318. Additionally, implant includes first tabs 324, 326 and second tabs 328, 330.

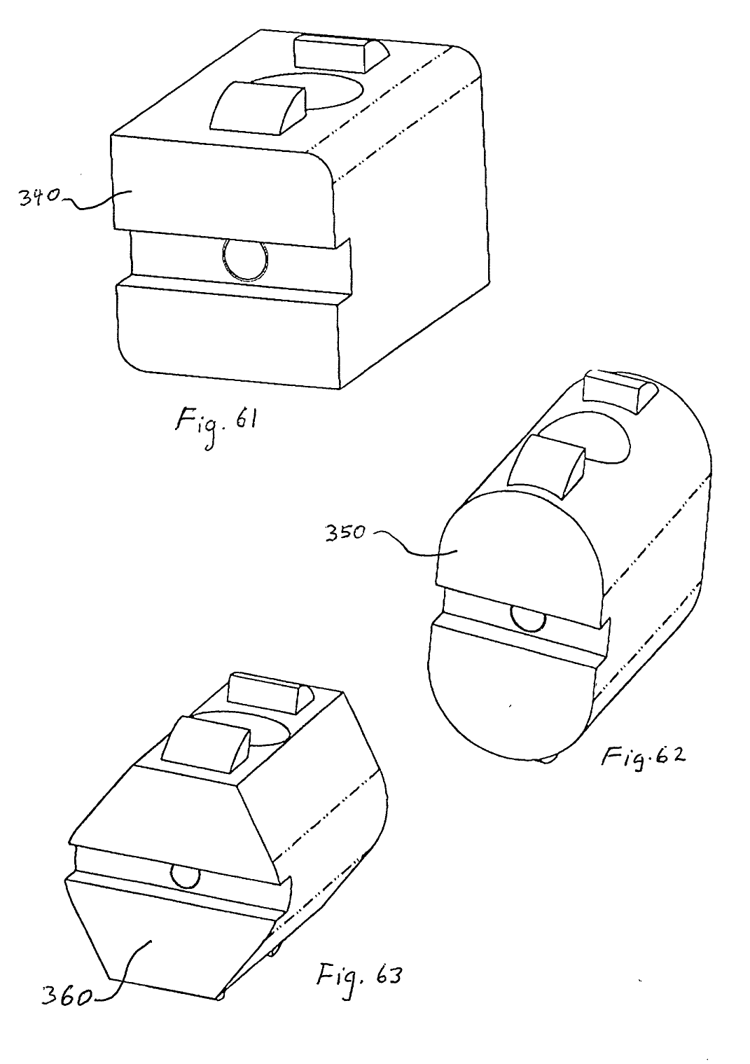

As best shown in FIGS. 61-63, various body portions other than cylindrical are within the contemplated scope of the present disclosure. These body portions may include a body portion 340, having a rectangular cross-section (FIG. 61), a body portion 350 having an oval cross-section (FIG. 62), a body portion 360 having a multi-sided cross-section (FIG. 63), etc. The embodiments disclosed in FIGS. 61-63 may obviously include structure similar or identical to that provided in previously described embodiments such as, for example, throughbores, installation slots, bore and all the various configurations and orientations of tabs.

It will be understood that various modifications may be made to the embodiments disclosed herein. For example, differing or alternate tab constructions may be provided on a single implant. Additionally, the various configurations may be combined on individual tabs. Therefore, the above description should not be construed as limiting, but merely as exemplifications of preferred embodiments. Those skilled in the art will envision other modifications within the scope and spirit of the claims appended hereto.

What is claimed is:

1. An intervertebral implant comprising:
a substantially cylindrical body portion having a first end and a second end; and
at least two tabs extending radially outward from the substantially cylindrical body portion, each of the at least two tabs being longitudinally displaced from the first and second ends.

2. An intervertebral implant according to claim 1, wherein the at least two tabs include a first tab and a second tab, the first tab being radially spaced approximately 180° about the substantially cylindrical body portion from the second tab.

3. An intervertebral implant according to claim 2, wherein the first tab is longitudinally spaced along the substantially cylindrical body portion from the second tab.

4. An intervertebral implant according to claim 1, wherein the substantially cylindrical body portion has a longitudinal axis and at least one throughbore defined in the substantially cylindrical body portion, the throughbore having a central axis which is substantially perpendicular to the longitudinal axis of the substantially cylindrical body portion.

5. An intervertebral implant according to claim 1, wherein said substantially cylindrical body portion has a maximum diameter, and each tab of the at least two tabs has a width less than or equal to the maximum diameter of the substantially cylindrical body portion.

6. An intervertebral implant according to claim 1, wherein said substantially cylindrical body portion defines an installation slot in one end thereof.

7. An intervertebral implant according to claim 4, wherein said substantially cylindrical body portion defines an installation slot in one end thereof and a bore extending between the slot and the throughbore.

8. An intervertebral implant according to claim 4, wherein the at least two tabs are radially spaced from the throughbore.

9. An intervertebral implant according to claim 1, wherein the at least two tabs include a pair of radially opposed first tabs and a pair of radially opposed second tabs.

10. An intervertebral implant according to claim 1, wherein each tab of the at least two tabs has a wedge-shaped surface.

11. An intervertebral implant according to claim 1, wherein each tab of the at least two tabs has a camming surface.

12. An intervertebral implant as claimed in claim 11, wherein each camming surface is flat.

13. An intervertebral implant as claimed in claim 11, wherein each camming surface includes opposed inclined camming surfaces.

14. An intervertebral implant according to claim 11, wherein each tab of the at least two tabs has a profile that defines a progressive camming surface.

15. An intervertebral implant according to claim 1, wherein each tab of the at least two tabs includes a threaded bone engaging surface.

16. An intervertebral implant according to claim 1, wherein the substantially cylindrical body portion defines a throughbore and each tab of the at least two tabs is an end of a plug positioned through the throughbore.

17. An intervertebral implant according to claim 1, wherein the substantially cylindrical body portion is tapered.

18. The intervertebral implant according to claim 1, wherein the implant is formed from a biocompatible material.

19. The intervertebral implant according to claim 18, wherein the implant is formed of bone.

20. The intervertebral implant according to claim 19, wherein the bone is animal bone.

21. The intervertebral implant according to claim 19, wherein the bone is human.

22. The intervertebral implant according to claim 19, wherein the bone is surface demineralized.

23. A method of installing an intervertebral implant between adjacent vertebrae comprising the steps of:
providing an intervertebral implant having a substantially cylindrical body portion and at least two tabs extending radially from the body portion;
forming a stepped bore in a portion of two adjacent vertebrae, the stepped bore having an enlarged diameter area and a reduced diameter area;
aligning the at least two tabs with a space defined between the adjacent vertebrae;
inserting the implant into the space a sufficient distance such that the at least two tabs are positioned adjacent the enlarged diameter area of the bore; and
rotating the implant to position the tabs within the enlarged diameter are of the bore.

24. An intervertebral implant comprising:
a body portion having a first end and a second end; and
at least two tabs extending radially outward from the body portion, each of the at least two tabs being longitudinally displaced from the first and second ends.

25. The intervertebral implant according to claim 24, wherein the body portion has a substantially rectangular cross-section.

26. The intervertebral implant according to claim 24, wherein the body portion has a substantially oval cross-section.

27. The intervertebral implant according to claim 24, wherein the body portion has a substantially multi-sided cross-section.
